(19) United States
(12) Patent Application Publication
(10) Pub. No.: US 2008/0245373 A1
(43) Pub. Date: Oct. 9, 2008

(54) ANTI SLIPPAGE ARM BOARDS

(75) Inventor: TERYLE L. KOUNKEL, Spirit Lake, IA (US)

Correspondence Address: MCKEE, VOORHEES & SEASE, P.L.C., 801 GRAND AVENUE, SUITE 3200, DES MOINES, IA, 50309-2721, US

(73) Assignee: BROWN MEDICAL INDUSTRIES, Spirit Lake, IA (US)

(21) Appl. No.: 11/697,055

(22) Filed: Apr. 5, 2007

(51) Int. Cl. A61F 5/37

(52) U.S. Cl. 128/877

(57) ABSTRACT

A support board for intravenous devices and the like that is nonslip in order to prevent the I.V. from slipping to and fro, and eventually out of the patient. The support board, commonly an arm board, is a splint having an upper surface with a foam cushion adhered to it, a lower surface with a nonskid material adhered to it and a cloth-like stockinette covering the entire splint which abrades against both surfaces without slipping to and fro.

### FIELD OF THE INVENTION

The field of this invention is support boards for I.V. devices, catheters and the like is commonly used in hospitals.

### BACKGROUND OF THE INVENTION

As is widely known, medicaments are frequently administered as a supplement through the employment of various devices used in conjunction with intravenous administration (I.V.) of fluids to patients. In particular, in order to minimize the amount of bolus injections given a patient, patients are often hooked up with an intravenous cathelon tube (see FIG. 3). In this way, various attachments can be made to the I.V. cathelon for administration of I.V. fluids such as glucose, saline, medicaments of various kinds, etc. Commonly, the devices use a pre-pierced, self-healing male adapter plug with a locking leur, which can be opened, attached to the catheter, and used for continuous I.V. administration.

Typically the various attachments that can be made to the I.V. cathelon for administration of I.V. fluids through the one common entry point are taped to an arm board or the like in order to ensure they stay in place and are not pulled away from the entry port during normal patient movement. Because the arm boards are in place for a long period of time, including during sleep, it is essential that they are comfortable at the interface of the patient's skin and the board. To assure comfort, some have used cushioned arm boards that are covered with a stockinette. While the stockinette covering enhances comfort at the pressure points against the skin, such stockinettes typically move easily to and fro with respect to the underlying support board they cover. This can and often does result in pulling and tugging against the I.V. device connection, often “unplugging” the device. As a result, it fails to function properly. In particular the medication that is being administered may not go into the patient intravenously as desired but may be spilled, necessitating a cleaning operation and a restart.

From the above description, it can be seen that there is a need to provide an I.V. support board that is nonslip, but at the same time provides comfort against the skin of the patient and which allows support of an I.V. device without running the risk of to and fro movement to “unplug” the I.V.

It is a primary objective of the present invention to solve the above need.

The method and means of accomplishing the above objective and of solving of the primary need mentioned will become apparent from the detailed description of the invention which follows hereinafter. The exact construction here shown is illustrative only. Put another way, the configuration can be changed and still achieve the invention results, i.e., anti-slip.

### BRIEF SUMMARY OF THE INVENTION

A support board for intravenous devices and the like that is nonslip in order to prevent the I.V. from slipping to and fro, and eventually out of the patient. The support board, commonly an arm board, is a splint having an upper surface with a foam cushion adhered to it, a lower surface with a nonskid material adhered to it and a cloth-like stockinette covering the entire splint which abrades against both surfaces without slipping to and fro.

### BRIEF DESCRIPTION OF THE DRAWINGS

FIG. 1 is a perspective view of the nonslip arm board of the invention.

FIG. 2 is a section along line 2-2 of the board of FIG. 1.

FIG. 3 shows the device of the present invention, worn upon a patient's arm and in location for I.V. administration.

### DETAILED DESCRIPTION OF THE PREFERRED EMBODIMENT

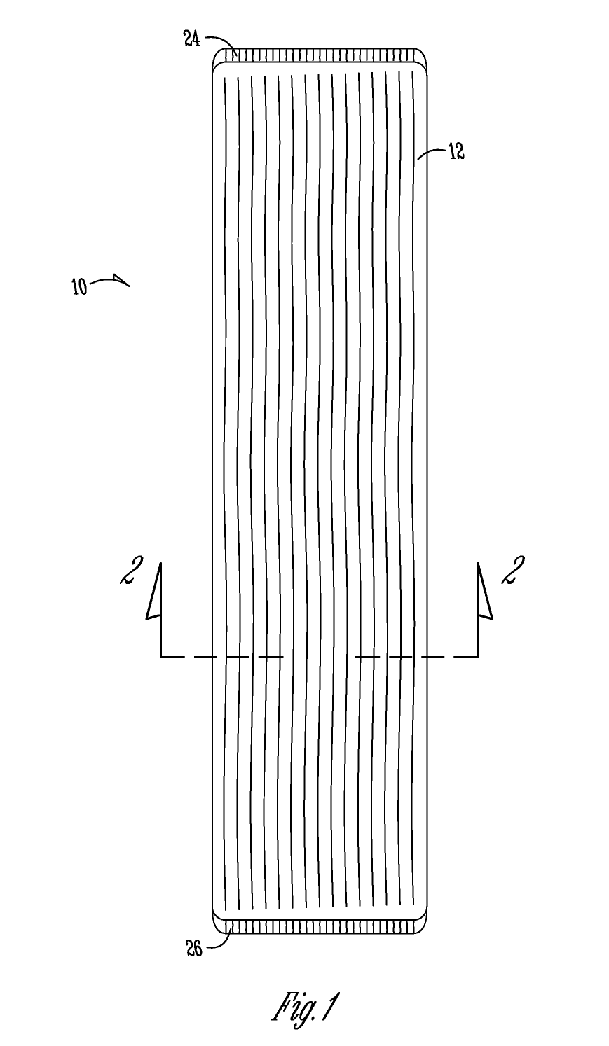

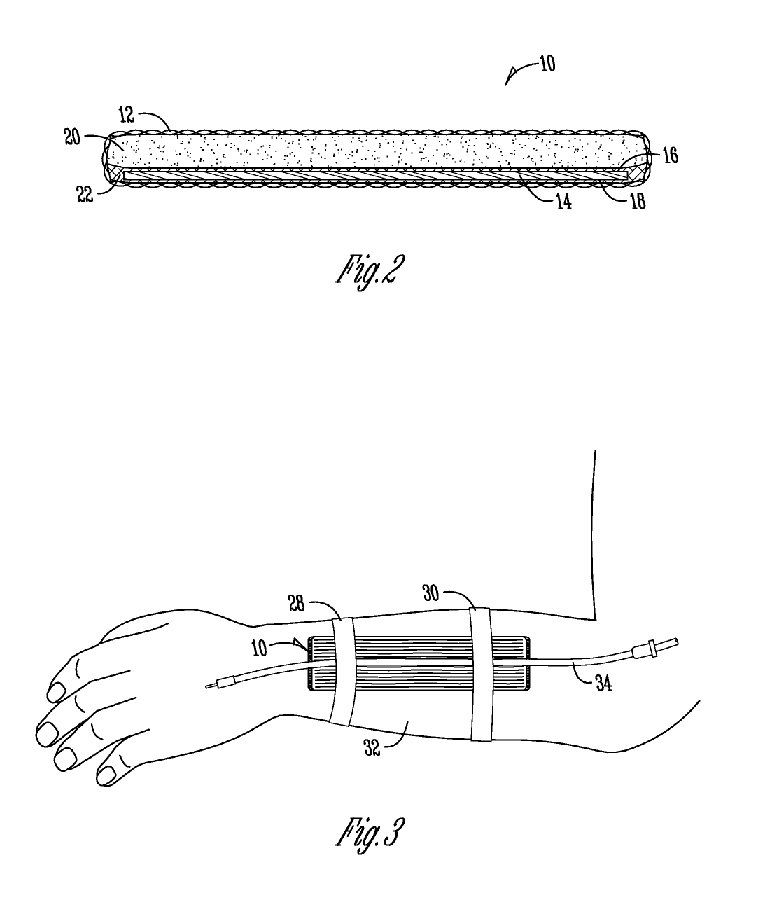

Looking at FIG. 1 there is shown a nonslip I.V. support board device 10 covered with a surrounding or sheathing stockinette 12. FIG. 2 shows the layers of the arm board support device 10. Centrally located is a splint 14 having an upper surface 16 and a lower surface 18 and adhered by a suitable adhesive material to upper surface 16 is a foam cushioning material 20. Similarly, adhered to the lower surface 18 of splint 14 is velour-like material 22 having a nonskid surface. Surrounding all of this in sheath-like fashion is a covering or stockinette 12 sewed at its respective ends as indicated by seams 24 and 26.

The stockinette 12 can be made of any suitable stretchable material, natural or synthetic fibers including cotton, rayon, nylon, and other polymeric base materials such as polypropylene, etc.

The foam cushion 20 can be made again of conventional materials, such as open cell and closed cell foams or rubbers. The exact adhesive selected is not critical with any naturally occurring or synthetic adhesive material that will at the same time effectively seal the foam material 12 to the splint 14 and the velour covering 22 to the splint can be used.

The splint itself can be aluminum or more commonly can be polycarbonate resin (PETG) such as Lexan®. It can, of course, be made of other materials such as PVC, PVS, PBS and alpha-olefins such as polypropylene/polyethylene or mixtures thereof.

The velour covering 22 can be of any suitable material that has a closely napped velvet-like exterior surface. One particularly suitable material is a flocked vinyl sheet that is commercially available from Adams Plastics. To produce the flocked vinyl, a manufacturer first produces a sheet of vinyl. This vinyl is then run through a machine which applies the flocking to the surface. The flocking material has previously been cut into small pieces. The flocking is put into a larger hopper, and stirred/fluffed about by the larger blower, so it is uniformly blowing about inside of the hopper. The vinyl sheet has adhesive applied to one surface, and the sheet is inserted through a slot into the hopper, exiting out the opposite side.

As the sheet of vinyl passes through the hopper, some of the flocking material sticks to the adhesive. The excess/unadhered flocking is blown off of the vinyl, and the adhesive is cured, finishing the process.

The sheet material is vinyl, and the flocking material is cotton. It goes without saying that other materials could be used for both, as well as different thickness/lengths of materials, etc.

In actual operation the device is taped by tape strips 28 and 30 around the arm 32 of a patient in order to support an I.V. device 34. The stockinette 12 will not slip around, to and fro, with respect to the underlying splint 14 because the foam cushion abrades against it and prevents movement on one surface and the velour-like backing 22 does the same for the other surface. This means the I.V. device 34 does not risk being pulled from its operating and I.V. administering position, risking loss of medication.

It can therefore be seen that the invention accomplishes at least all of its objectives.

What is claimed is:

1. An I.V. support board that is nonslip, comprising:
a splint having a length, an upper surface, and a lower surface;
a foam cushion material adhered to at least one of said surfaces;
a material having a nonskid surface adhered to either the foam or the other surface; and
a cloth like stockinette covering surrounding the entire splint which can abrade against the foam cushion material on one surface and against the nonskid material on the other surface to provide a nonslip I.V. support board.

2. The support board of claim 1 wherein the splint is made from a material selected from the group consisting of wood, plastics, Lexan®, polycarbonate resin, PVC, PVS, PBS and alpha-olefin polymers.

3. The support board of claim 1 wherein the nonskid surface material is a closely napped cloth material.

4. The support board of claim 3 wherein the nonskid surface material is velour.

5. The support board of claim 1 wherein the stockinette is a synthetic material.

6. The support board of claim 1 wherein the stockinette is a natural material.

7. An I.V. support board that is nonslip, comprising:
a splint having a length, an upper surface, and a lower surface;
a foam cushion material adhered to one of said surfaces;
a material having a nonskid surface adhered to the other surface; and
a cloth like stockinette covering surrounding the entire splint which can abrade against the foam cushion material on one surface and against the nonskid material on the other surface to provide a nonslip I.V. support board.

8. The support board of claim 7 wherein the splint is made from a material selected from the group consisting of wood, plastics, Lexan®, polycarbonate resin, PVC, PVS, PBS and alpha-olefin polymers.

9. The support board of claim 7 wherein the nonskid surface material is a closely napped cloth material.

10. The support board of claim 9 wherein the nonskid surface material is velour.

11. The support board of claim 7 wherein the stockinette is a synthetic material.

12. The support board of claim 7 wherein the stockinette is a natural material.

13. A method of providing an I.V. support board that is nonslip, comprising:
providing a splint having a length, an upper surface and a lower surface;
adhering a foam cushion to one of said surfaces;
adhering a material having a nonskid surface to the other surface; and
surrounding the entire splint with a cloth-like stockinette covering which can abrade against the foam cushion material on one surface and against the nonskid material on the other surface to provide a nonslip I.V. support board.
